(19) United States
(12) Patent Application Publication
(10) Pub. No.: US 2013/0131151 A1
(43) Pub. Date: May 23, 2013

(54) METHOD FOR PHOTOPOLYMERIZING HYDROGEL USING X-RAY IRRADIATION

(71) Applicants: YEU KUANG HWU, TAIPEI (TW); S JA TSENG, TAIPEI (TW)

(72) Inventors: YEU KUANG HWU, TAIPEI (TW); S JA TSENG, TAIPEI (TW)

(73) Assignee: INSTITUTE OF PHYSICS, ACADEMIA SINICA, TAIPEI (TW)

(21) Appl. No.: 13/405,055

(22) Filed: Feb. 24, 2012

Related U.S. Application Data
(60) Provisional application No. 61/560,936, filed on Nov. 17, 2011.

(51) Int. Cl. A61K 47/32; C08L 33/14; A61K 31/711; C08K 5/42; A61K 31/7105; C08K 5/3465; C08K 3/02

(52) U.S. Cl. 514/44 R; 522/108; 522/81; 522/72; 514/772.3

(57) ABSTRACT

A method for preparing a hydrogel includes the steps of injecting a precursor with at least two alkene groups into a predetermined portion, injecting at least one specie into the predetermined portion, and performing an X-ray irradiation on the predetermined portion to induce a polymerization reaction of the precursor to form a porous hydrogel with the specie embedded inside the porous hydrogel. In one embodiment of the present invention, the specie is selected from the group consisting of nucleic acid and adhesion agent.

### BACKGROUND OF THE INVENTION

1. Technical Field

The present invention relates to a method for photopolymerizing hydrogel using X-ray irradiation, and more particularly, to a method for photopolymerizing hydrogel in vivo using X-ray irradiation.

2. Background

The fabrication of 3D biomaterial implants has been the subject of much research due to their potential for a broad range of biomedical applications. Such 3D implants can serve as temporary scaffolds and promote cell reorganization and the formation of biofunctional substitutes. Hydrogel has also attracted much attention particularly for use in local delivery of matrix-encapsulated proteins and nucleic acids to chondrocytes, fibroblasts, vascular smooth muscle cells, osteoblasts, neural precursor cells and stem cells for regenerative therapies, since many biophysical properties of Hydrogel are similar to those of soft biological tissues. Hydrogel can also be used to obtain flexible 3D formations, mechanical stability and a good tissue culture environment for biomedical applications.

Notwithstanding the above-mentioned positive points, a serious obstacle remains to be solved for the wide application of hydrogel: the need for accurate administration in vivo. Inserting a solid material, even gel-like, into a live system is difficult without undesirable surgery. While many different approaches exploiting a phase change after injection from a more easily handled liquid to a gel have been explored. Hydrogel is one of the most biocompatible materials exhibiting such a phase change. Of particular interest in this context, beyond passive implantation, is the local activation of cell-material interactions.

The practical implementation of this approach requires injectable precursors and an initiator that stimulates polymerization at precise locations deep in the tissue. Low-viscosity precursor hydrogel solutions can be quite easily injected by syringe with the appropriate mixture of cells and bioactive factors, and placed at the desired 3D location, to be then polymerized by the initiator. Accurate initiator administration is a much more serious problem. Typical polymerization initiators include heat, chemicals, mechanical factors, ultrasound and photons, all of which are difficult to accurately administer in vivo. Recently, it was found that glucose oxidation and Fe²⁺ generate hydrogel within minutes at room temperature and ambient-pressure oxygen. This was found to yield cellular encapsulation into hydrogel scaffolds, but the reaction rate must be improved for in vivo administration. Another approach, shear thinning, produced gelation in vivo, but also requires administration in vivo via a long circulation path, beyond local injection, for which mechanical factors cannot be easily controlled and monitored.

Photopolymerization by visible or ultraviolet (UV) light exploiting photoinitiators to produce free radicals, which subsequently initiate polymerization through active sites on macromeric chains, could solve the problem. The space and time characteristics of the polymerization process can be controlled by the shape and intensity of the light beam as well as by the illumination time. The polymerization rate can be high enough to produce hydrogel with a short exposure (seconds to tens of seconds).

Photopolymerization is already used for biomedical hydrogel production. Under standard protocols, the acrylate-terminated monomer undergoes photopolymerization by exposure to light in the presence of appropriate photoinitiators. It has been shown that UV and visible light polymerize hydrogel in vivo for cell encapsulation applications requiring high biocompatibility during the polymerization process without adversely affecting the encapsulated cells. Another example of successful in vivo application is transdermal photopolymerization, again using UV and visible light, which opens the door to minimally invasive hydrogel implantation. However, many photoinitiators, particularly those with UV or visible absorption, have some water solubility and cytotoxicity problems. Furthermore, light scattering and absorption limit the use of UV or visible photopolymerization where accurate local control of the implantation is required. These problems, linked to the administration depth and to shadowing, stimulated the development of light-independent encapsulation systems for cell-laden scaffolds.

Thus far, none of the above approaches has been fully satisfactory in accurately polymerizing the scaffold leading to the desired 3D shapes in vivo. Also note that a high polymerization rate in vivo is required for the hydrogel to maintain its shape, location and functions without complications due to the biological response.

### SUMMARY

One aspect of the present invention provides a method for photopolymerizing hydrogel in vivo using X-ray irradiation.

A method for preparing a hydrogel according to this aspect of the present invention comprises the steps of injecting a precursor with at least two alkene groups into a predetermined portion, injecting at least one specie into the predetermined portion, and performing an X-ray irradiation on the predetermined portion to induce a polymerization reaction of the precursor to form a porous hydrogel with the specie embedded inside the porous hydrogel. In one embodiment of the present invention, the specie is selected from the group consisting of nucleic acid and adhesion agent.

The foregoing has outlined rather broadly the features and technical advantages of the present invention in order that the detailed description of the invention that follows may be better understood. Additional features and advantages of the invention will be described hereinafter, and form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

The objectives and advantages of the present invention are illustrated with the following description and upon reference to the accompanying drawings in which:

FIG. 1 illustrates a schematic explanation of hydrogel photopolymerization by X-ray irradiation in a live animal according to one embodiment of the present invention.

FIG. 2A illustrates a synthetic scheme for the preparation of PEG-DA-based hydrogel according to one embodiment of the present invention, wherein the precursor solutions of PEG-DA—either pure or mixed with heparin or PEI/nucleic acids (plasmid DNA or siRNA) nanoparticles—are exposed to an X-ray beam.

FIG. 2B illustrates an FTIR spectra of PEG-DA and PEG-DA-based hydrogel after different irradiation times according to one embodiment of the present invention.

FIG. 2C illustrates a DA percentage measured by ¹H-NMR in the PEG DA-based hydrogel after different irradiation times according to one embodiment of the present invention, wherein the results for this figure as well as for those of FIGS. 3 to 7 are presented as mean±one standard deviation for measurements on n=3 samples.

FIG. 3A illustrates a photograph of the clear PEG DA-based hydrogels produced in a transparent tube (10 mm diameter, 5 mm height) according to one embodiment of the present invention. The two shapes were obtained by rotating the tube during irradiation with an X-ray beam passing through a square mask (top) or a mask with a smaller top opening (bottom). The PEG DA-based hydrogel was immersed in a PBS solution and imaged by a standard inverted microscope (upper left image). The lower left image refers to the control specimen.

FIG. 3B illustrates an SEM (scanning electron microscope) micrograph of the X-Y plane of the PEG DA-based hydrogel according to one embodiment of the present invention, wherein the scale bar is 20 μm.

FIG. 3C illustrates a weight loss and FIG. 3D illustrates a swelling ratio of the hydrogel as a function of the time spent after polymerization in a PBS solution at 37° C. according to one embodiment of the present invention.

FIG. 4A illustrates a cytotoxicity of PEG DA according to one embodiment of the present invention;

FIG. 4B illustrates hydrogels photopolymerized by X-ray irradiation according to one embodiment of the present invention. The normalized HT-1080 cell viability was calculated by comparing the absorbance in the different specimens to that of untreated cells.

FIG. 5A and FIG. 5B show morphology of human HT-1080 fibroblasts with 1 day post seeding on PEG DA-based hydrogel and hydrogel with heparin inclusion respectively according to one embodiment of the present invention, wherein insets show higher magnification images of the areas marked with squares. Scale bars: 200 μm.

FIG. 6A illustrates a cytotoxicity of PEG DA-based hydrogels with heparin inclusion at different concentrations according to one embodiment of the present invention. The normalized HT-1080 cell viability was calculated by comparing its absorbance to that of untreated cells.

FIG. 6B illustrates an in vitro swelling ratio of the hydrogel as a function of the time spent, after polymerization, in a PBS solution at 37° C. according to one embodiment of the present invention.

FIGS. 7A and 7B illustrate the transmission electron micrograph (TEM) showing both kinds of PEI/plasmid DNA and PEI/siRNA nanoparticles (scale bar, 250 nm) respectively according to one embodiment of the present invention.

FIG. 8A shows a gene expression mediated by hydrogels of PEI/plasmid DNA nanoparticles according to one embodiment of the present invention. Confocal microscopy images (scale bar, 50 μm) show normal HT-1080 cells transfected, wherein the upper panel of FIG. 8A refers to control cells without any treatment.

FIG. 8B shows fluorescence intensity obtained from FIG. 4A showing the kinetics of fluorescence intensity of EGFP expression caused by PEG DA-based hydrogel inclusion with PEI/plasmid DNA nanoparticles.

FIG. 9A shows a gene silencing process mediated by our hydrogel of PEI/GFP-22 siRNA nanoparticles according to one embodiment of the present invention. Confocal microscopy images (scale bar, 50 μm) show recombinant HT-1080 cells transfected, wherein the upper panel of FIG. 9A refers to control cells without any treatment.

FIG. 9B shows a fluorescence intensity obtained from FIG. 9A showing the kinetics of fluorescence intensity of EGFP silencing caused by PEG DA-based hydrogel inclusion with PEI/GFP-22 siRNA nanoparticles according to one embodiment of the present invention.

FIG. 10 shows optical photomicrographs of histological sections with the precursor solution, the hydrogel and its surrounding tissue prepared 8 days after the photopolymerization according to one embodiment of the present invention. Areas with strong pink signals are infiltrating neutrophils in subcutaneous mouse tissue (marked by the asterisk).

FIG. 11 shows a hydrogel formation by 30 seconds of X-ray irradiation in the presence of bare Au nanoparticles according to one embodiment of the present invention. The DA precursor diminished more rapidly during irradiation for increasing quantities of nanoparticles. The control (black) is pure precursor solution.

### DETAILED DESCRIPTION

The inventors tested an alternate method and demonstrated its capability to produce accurate 3D hydrogel implants without surgery. The key factor was polymerization of PEG diacrylate (DA) by short-wavelength, high penetration irradiation, wherein the process was substantially stimulated in situ by X-rays. These tests open the way to a potentially very flexible approach, since X-rays can be locally controlled so that the implant could in principle be “written” in 3D with excellent accuracy. Similar performances were demonstrated for X-ray lithography by combining multi-directional irradiation and variable masks; this enabled in particular the accurate fabrication of 3D structures such as photonic crystals. Since hydrogels are extensively used for the local delivery of proteins and other similar applications, the inventors also tested procedure to obtain hydrogel containing factors for gene delivery.

The inventors selected PEG DA-based hydrogel for several reasons. The PEG segment of PEG DA is non-immunogenic, non-toxic, and highly hydrophilic. The use of PEG for drugs was demonstrated to be safe and effective in many US Food and Drug Administration-approved therapeutic procedures. PEG is also extensively used as a coating substance to obtain biomaterial surfaces that resist non-specific protein adsorption and cell adhesion.

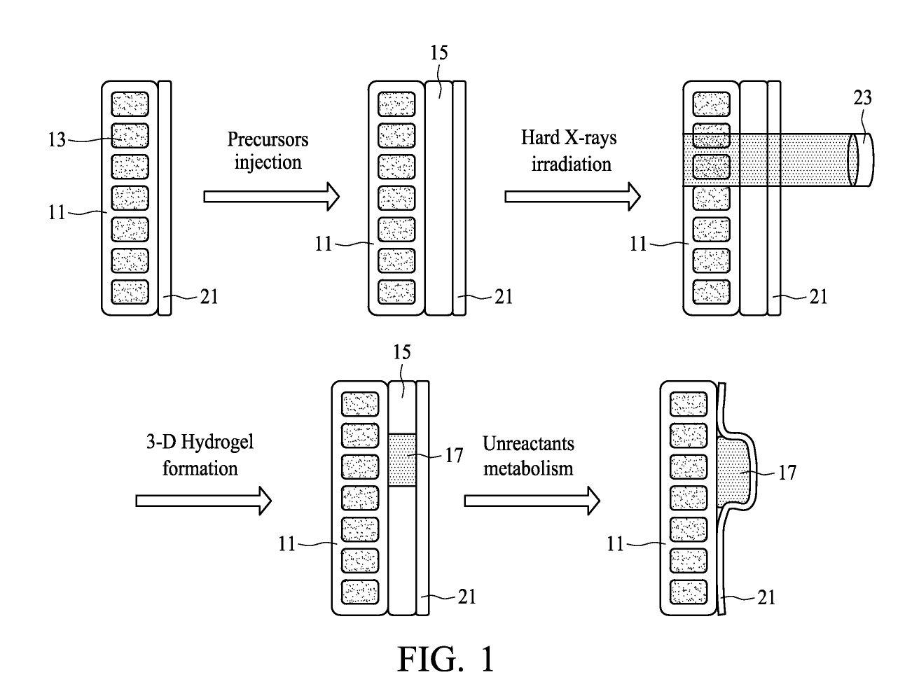

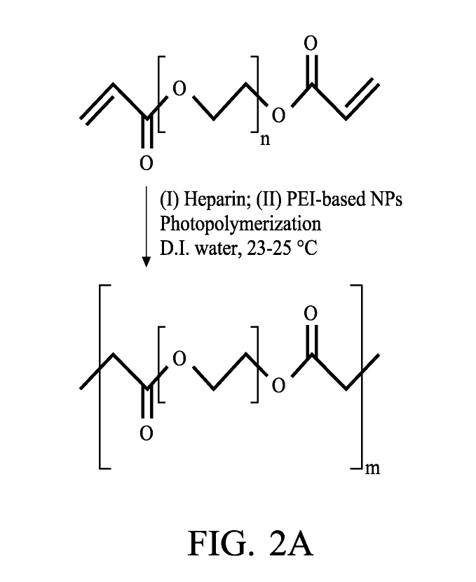

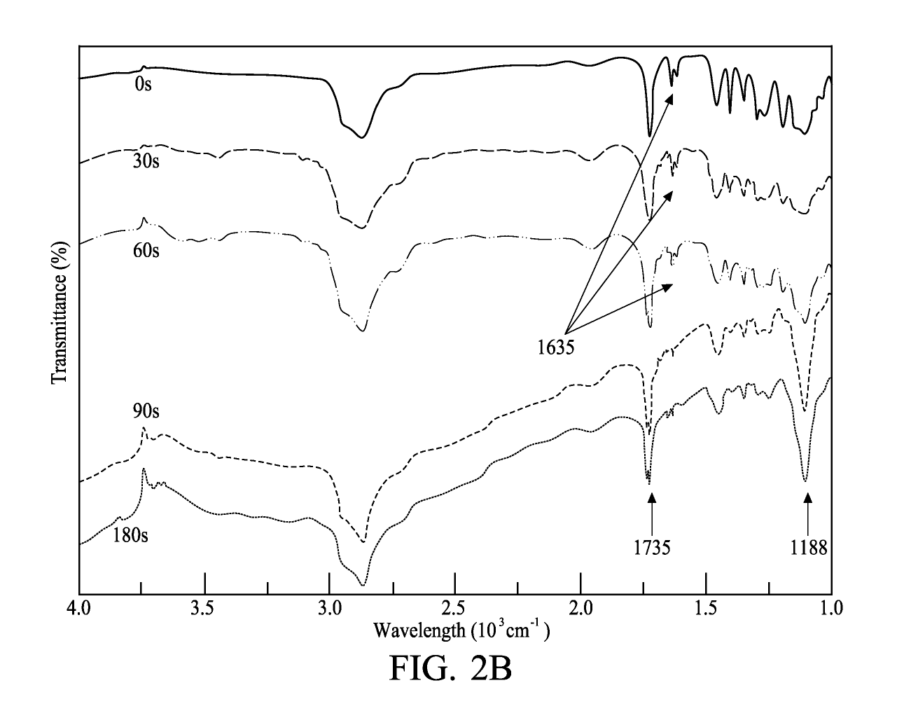

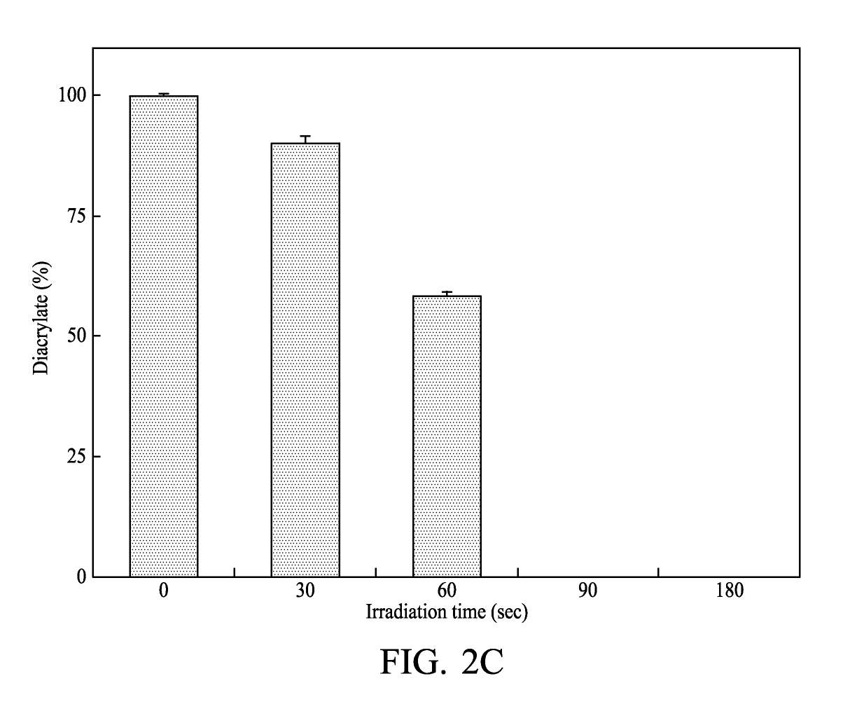

The approach schematically presented in FIG. 1 and FIG. 2 does not use chemical polymerization initiators. In one embodiment of the present invention, a precursor 15 with at least two alkene groups is injected into a predetermined portion inside a live system, wherein the predetermined portion can be between a skin 21 and blood vessel 11 with tissue 13. Subsequently, an irradiation is performed by using an X-ray 23 on the predetermined portion to induce a polymerization reaction of the precursor 15 to form a porous hydrogel 17. In one embodiment of the present invention, at least one specie is injected into the predetermined portion before the X-ray irradiation, and the specie is embedded inside the porous hydrogel after the polymerization reaction, wherein the specie is selected from the group consisting of nucleic acid and adhesion agent. In one embodiment of the invention, the precursor has a number average molecular weight of 258, 575, 700, 2000 or 60000.

The inventors first performed a subcutaneous injection (SC) in mice of the precursor solution including PEG DA and PBS (phosphate buffered saline) at the optimal concentration ratio. The exposure to X-rays triggered the in situ polymerization and hydrogel formation. The remaining non-polymerized solution was then physiologically eliminated from the subcutaneous tissue. The testing was extended to the photopolymerization of PEG DA-based hydrogels with heparin or PEI/DNA and PEI/siRNA nanoparticles and to the corresponding properties. The tests also identified measures that can increase the polymerization rate and reduce the risk of radiation effects. Such measures are based on the addition of Au nanoparticles in the precursor solution and could be improved in the future from the use of microfocused X-rays and from the development of even more X-ray sensitive biopolymers. With such improvements, the radiation dose could be reduced sufficiently to allow cell encapsulation procedures.

To assess the ability of PEG DA to polymerize under X-ray irradiation (FIG. 2A), the products are characterized by FTIR (Fourier transform infrared) measurements to identify their functional groups (FIG. 2B). The adsorption peaks of unpolymerized PEG DA are at 1635 cm⁻¹ (CC stretching, alkene), 1735 cm⁻¹ (CO stretching, ester) and 1188 cm⁻¹ (CO stretching, ester), indicating the presence of alkenes and ester. As the X-ray irradiation time increased, the alkene peak intensity decreased since the alkenes changed to hydrogel ethyl groups. The hydrogel synthesis was quantified by measuring the percentage of diacrylate with ¹H-NMR as a function of the irradiation time. The results are shown in FIG. 2C and reveal a progressive decrease, reaching a negligible value after 90 seconds, showing that complete polymerization was reached with 90 seconds irradiation. In this specific case, after 60 seconds of irradiation the polymerization of PEG-DA in D.I. water was not complete, as the DA percentage was 58.1%.

Compared to other hydrogel polymerization approaches, such as UV-induced polymerization and chemical crosslinking, the conversion rate by X-rays is quite reasonable. Note that this rate is limited by the need to use a safe X-ray dose. The dose, however, can be reduced by increasing the conversion rate; this appears feasible using materials with greater X-ray sensitivity and/or micro-focused X-rays that can increase the local intensity at selected points by orders of magnitude.

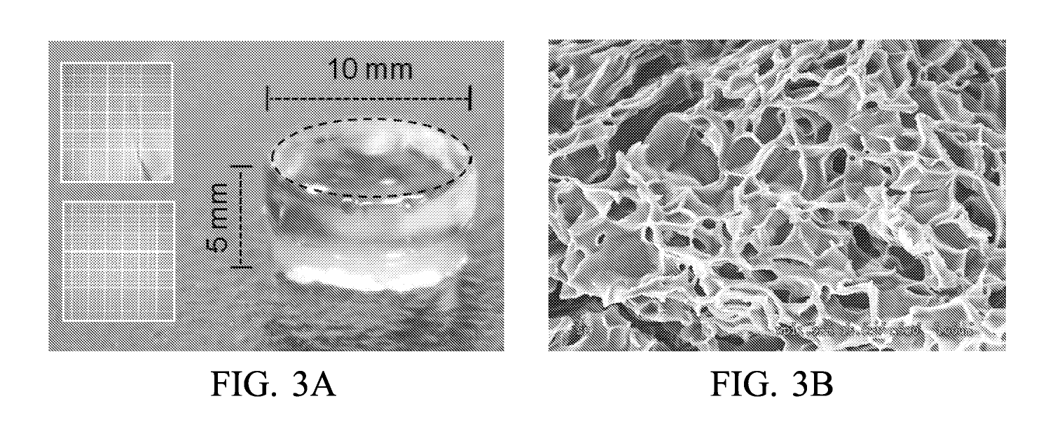

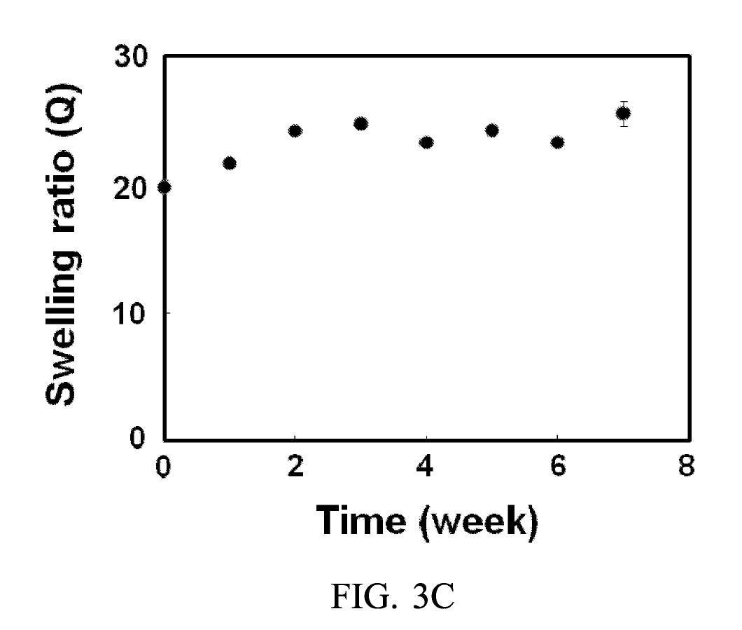

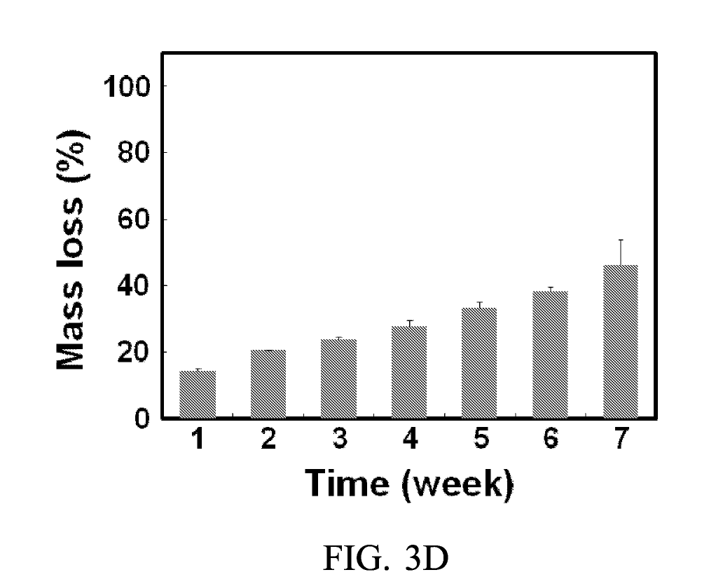

To evaluate the stability of the polymerized hydrogels, their weight loss (%) is measured over time in PBS solution at 37° C. For the assay, the hydrogels are produced in porous transparent cylinder containers (10 mm diameter, 5 mm height) (FIG. 3A and FIG. 3B). After 7 weeks, the mass loss was 47% (FIG. 3C), probably due to biodegradation of ester bonds. The mass loss was linear with time over 7 weeks, indicating that the mechanism for hydrogel degradation is surface erosion. We analyzed in parallel the swelling ratio change with time that indicates changes in the hydrogel physical and chemical structure. FIG. 3D shows typical results revealing an almost constant swelling ratio during hydrogel degradation.

Compared to other polymerization approaches, X-ray synthesized hydrogel is quite stable in terms of long-term weight loss and swelling ratio. This can be attributed to the less complex and uniform chemistry involved in the polymerization process.

### In Vitro Cytotoxicity of PEG DA and PEG DA-Based Hydrogels

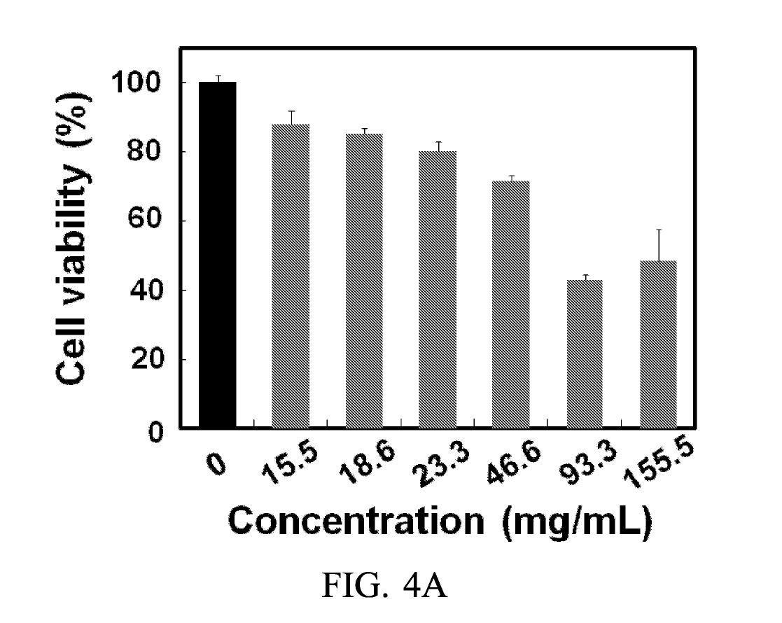

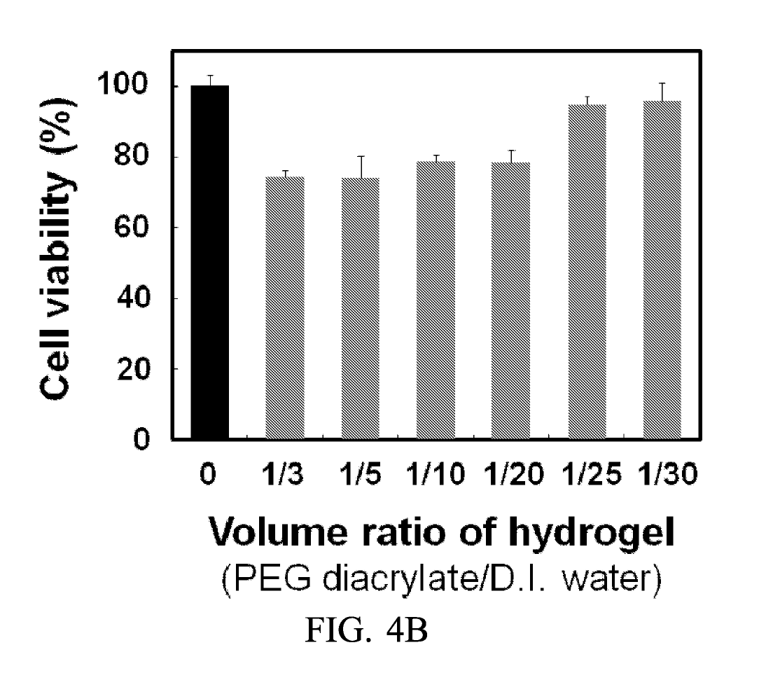

The testing was extended to the effects of the hydrogel on human HT-1080 fibroblast cells. The cell viability was determined by the MTS assay after 24 hours incubation with PEG DA at different concentrations, using untreated cells as the reference. PEG DA with 15.5, 18.6 and 23.3 mg mL⁻¹ concentration resulted in an average cell viability of greater than 80% (FIG. 4A). Higher concentrations, 93.3 and 155.5 mg mL⁻¹, decreased the viability to 42% and 48%, respectively. In contrast to the precursor PEG DA solution, polymerized hydrogels are essentially nontoxic if the precursor solution concentration is reasonably low. The results show that hydrogel from precursor solutions with PEG DA/D.I. water volume ratios 1/25 and 1/30 (PEG DA concentrations: 18.6 and 15.5 mg mL⁻¹) did not affect the cell viability (FIG. 4B). Our relative cytotoxicity values are comparable to those of other authors. The cell viability dropped from 95% to 74% when the volume ratio increased to 1/3 (PEG DA concentration: 155.5 mg mL⁻¹).

The toxicity tests are of course essential for the practical applications of the procedure. In our case, it is insufficient to merely consider the toxicity of the precursors (which are certified biomaterials) and of the hydrogel: one must also analyze their combined effects. Our testing verified that the X-rays did not alter the precursor or the hydrogel and produced no toxicity effect.

### Effects of Heparin Inclusion in Hydrogels

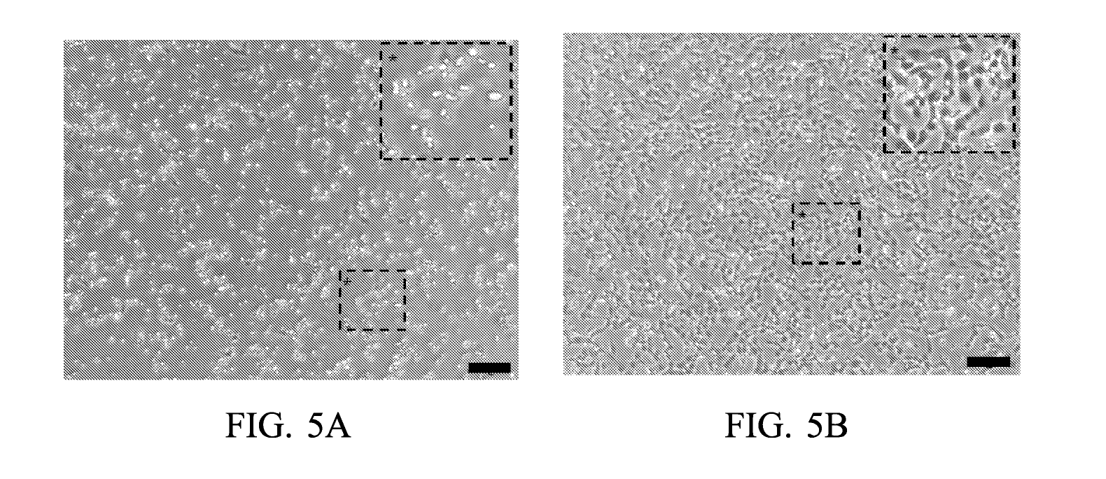

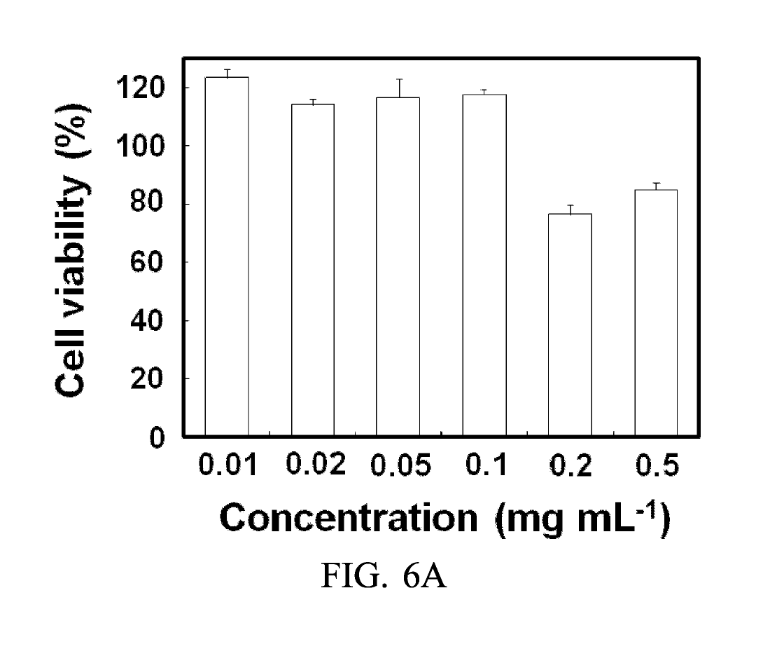

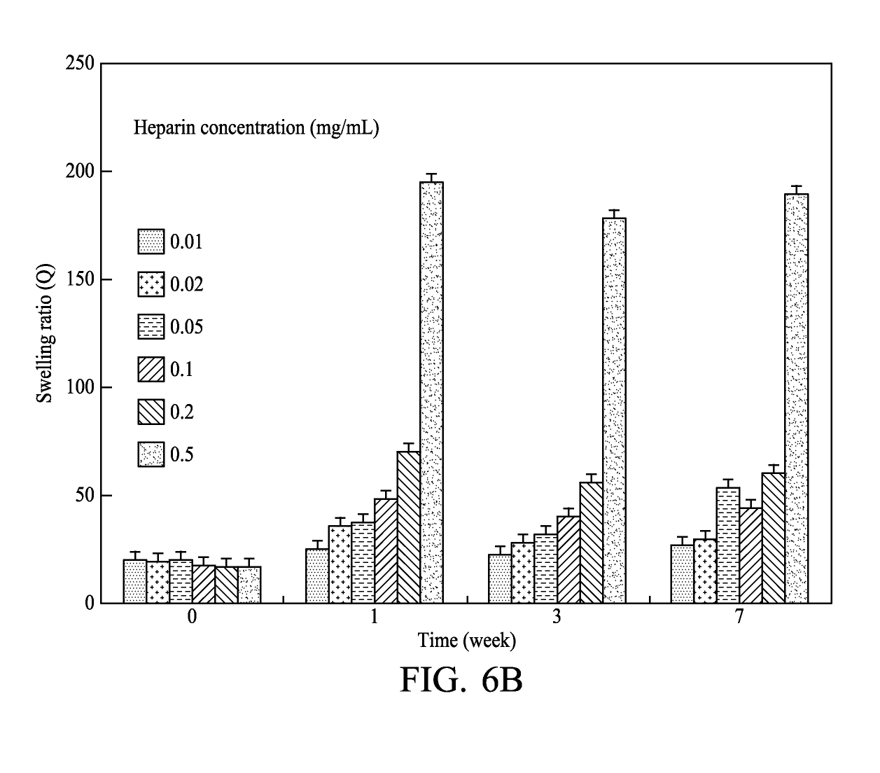

The adhesion of cells to the hydrogel is an important mechanical property that could affects its application. In our tests, we found that the HT-1080 cells on our PEG DA-based hydrogels had an abnormal spherical morphology (FIG. 5A). To deal with this problem, we used hydrogels with heparin to improve the cell adhesion (FIG. 5B). Such treatment had an unexpectedly positive impact on the cytotoxicity except for very high heparin concentrations: the cell viability increased as the concentration increased from 0.01 to 0.1 mg mL⁻¹ (FIG. 6A), whereas the cell viability decreased as concentration increased from 0.2 to 0.5 mg mL⁻¹. The swelling ratio increased with the heparin concentration (FIG. 6A). This is reasonable since heparin included in scaffolds leads to the absorption of relatively large quantities of water and improves the water contact angle on the scaffold surfaces. Thus, heparin inclusion increases the hydrogel volume (FIG. 6B) and also improves the cell adhesion.

### Nucleic Acid Transfection by Inclusion of PEI-Based Nanoparticles in Hydrogels

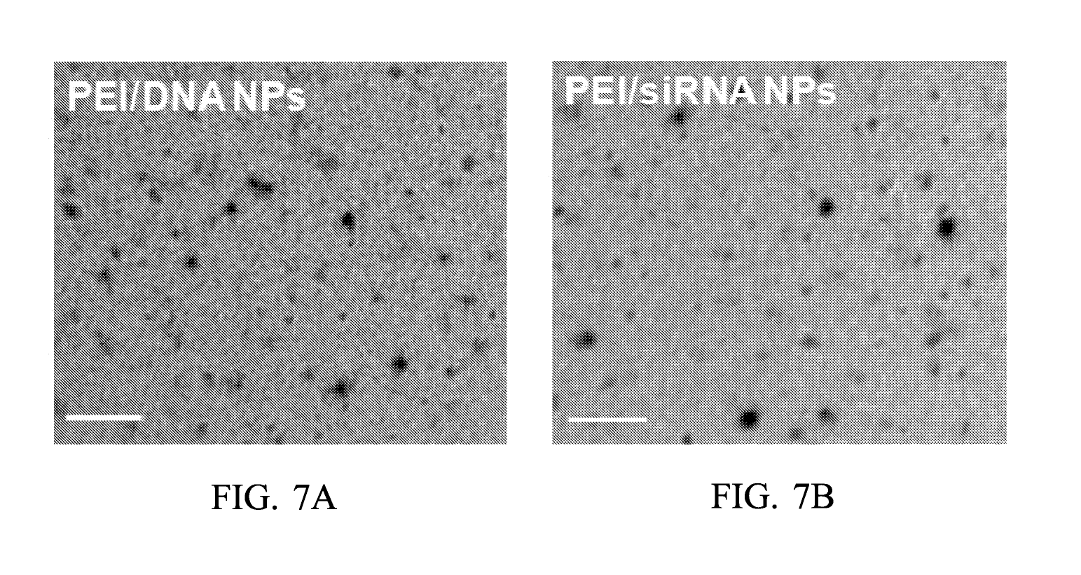

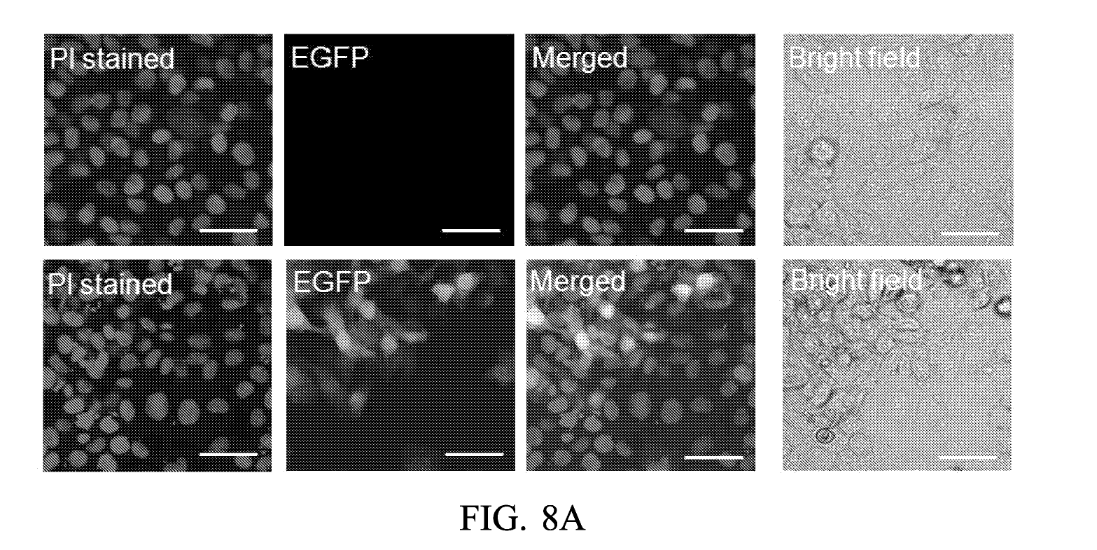

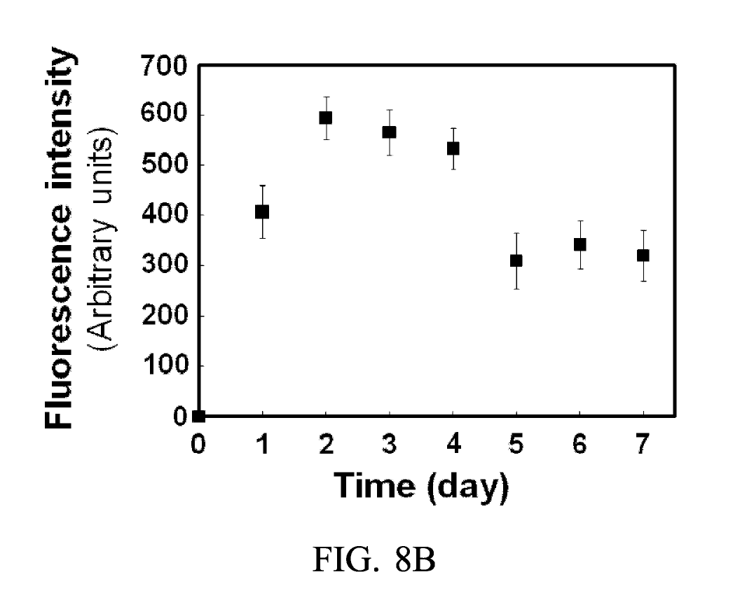

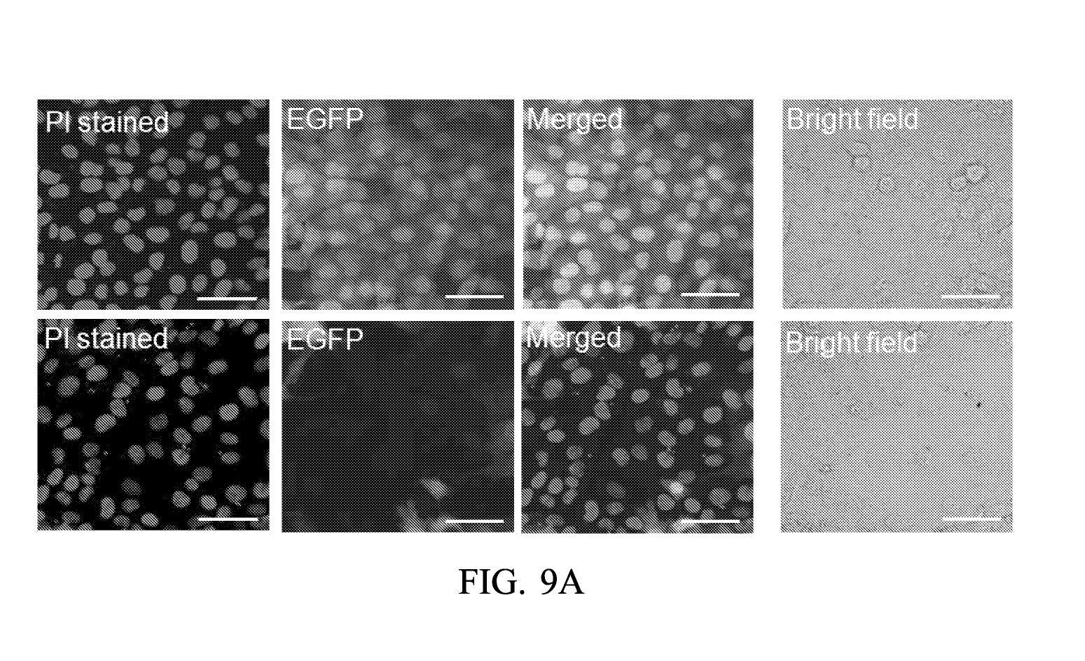

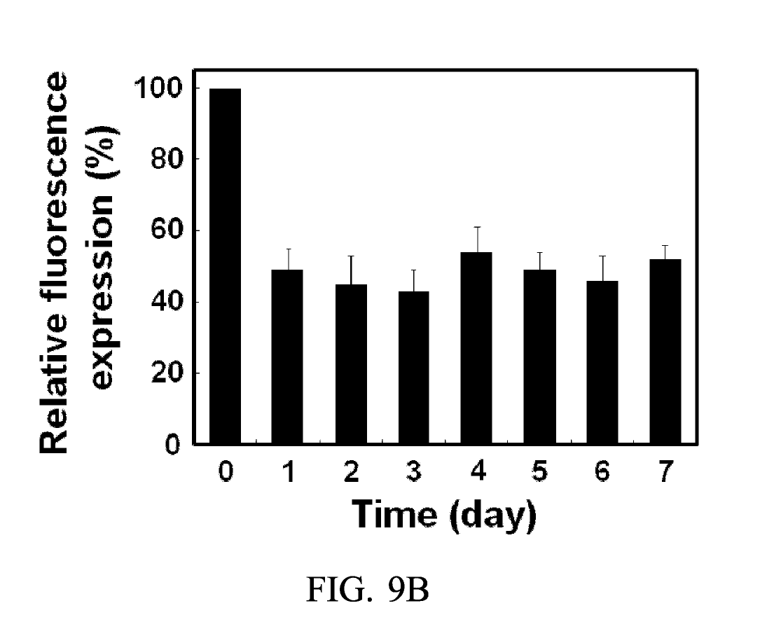

We tested the local delivery of plasmid DNA or small interfering RNA (siRNA) in nanoparticles by using PEI, which is widely exploited as a nucleic acid carrier. We found that the use of PEG DA-based hydrogel with PEI/plasmid DNA nanoparticles (FIG. 7A) leads to plasmid DNA expression; conversely, hydrogel with PEI/siRNA nanoparticles (FIG. 7B) silenced the target gene for gene knockdown. The results are shown in FIGS. 7 and 8; specifically, FIG. 8A refers to plasmid DNA delivery from hydrogel with inclusion of PEI/plasmid DNA nanoparticles. Our hydrogel delivered enough PEI/plasmid DNA nanoparticles to express EGFP over 1 week (FIG. 8B). FIG. 9A shows a significantly reduced EGFP expression in cells treated with PEG DA-based hydrogel including PEI/siRNA nanoparticles. Quantitatively, the eventual EGFP expression was less than 50% of that for control (no treatment) cells (FIG. 9B). Both gene expression and gene silencing were constant for 7 days, demonstrating that the nanoparticles were steadily released by the degraded hydrogel. These effects could be useful for tissue engineering and therapeutic procedures. In one embodiment of the invention, the nucleic acid carrier is the extracellular matrix selected from the group consisting of heparin, alginate, collagen, hyaluronan and the combination thereof.

### In Vivo Hydrogels Photopolymerization and Biocompatibility

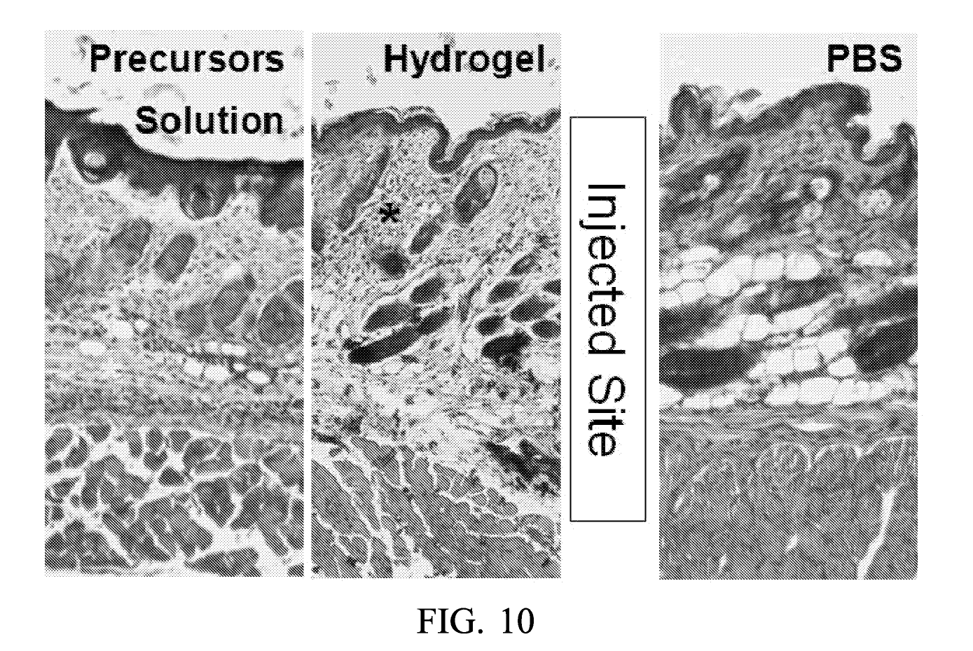

FIG. 8 shows the results of biocompatibility tests of our hydrogel: histological images of mouse subcutaneous tissue collected at day 8 and stained by H&E. There was no indication of epithelial erosion and a relatively small number of unusual neutrophil infiltration sites (marked by asterisks in FIG. 10). These biocompatibility results compare favorably with those of other authors.

### Enhancing the X-Ray Sensitivity by Gold Nanoparticle Inclusion

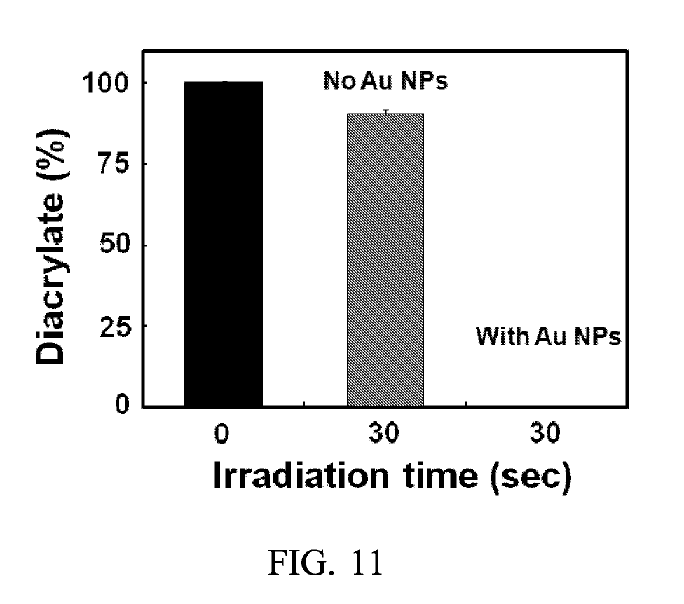

To decrease the X-ray dosage, we incorporated 20 mM bare gold nanoparticles of 15 to 20 nm diameter in the PEG DA solution before performing the x-ray irradiation tests. As a high Z material, Au strongly absorbs hard X-rays and can be expected to enhance their effects. The nanoparticle shape optimization can further increase this effect. In our tests, the precursor with gold nanoparticles was completely polymerized within 30 seconds, 3 times faster than without nanoparticles (FIG. 11). This effect may not be merely due to the increased X-ray absorption but also due to effects such as the increased production of radiochemicals. The success of these tests is quite important since gold nanoparticles are biocompatible and capable of performing many additional functions with surface modification. Combined with the future improvements brought by multi-direction irradiation and X-ray microfocusing, such gold nanoparticles could further increase the conversion efficiency and the process flexibility.

### EXPERIMENTAL

### Materials

PEG DA (Mn=700) and branched poly(ethylene imine) (PEI, Mw=25,000) were purchased from Aldrich (Milwaukee, Wis.). Heparin sodium (100 KU) and phosphate-buffered saline (PBS, pH 7.4) were purchased from Sigma Co. (St. Louis, Mo.). CellTiter 96® AQueous one solution cell proliferation assay systems for the MTS assay were purchased from Promega (Madison, Wis.). The plasmid DNA (pEGFP-N2, 4.7 kb, coding an enhanced green fluorescence protein reporter gene) was purchased from Clontech (Palo Alto, Calif.). pEGFP-N2 was amplified using DH5α and purified by Qiagen Plasmid Mega Kit (Germany) according to the manufacturer's instructions. The purity of plasmids was analyzed by gel electrophoresis (0.8% agarose), while their concentration was measured by UV absorption at 260 nm (Jasco, Tokyo, Japan). The siRNA duplex targeting enhanced green fluorescence protein (EGFP, GFP-22 siRNA) was purchased from Qiagen (Germany), and the siRNA sense sequence was EGFP, 5′-GCAAGCUGACCCUGAAGUUCAUdTdT-3′.

### Fabrication of 3-D PEG DA-Based Hydrogels by X-Ray Irradiation

The experiments were performed on Beamline 01A at the National Synchrotron Radiation Research Center (BL01A NSRRC, Hsinchu, Taiwan). Six silicon wafers (thickness: 550 μm) were used to reduce the dose rate from 5.10 kGy s⁻¹ to 110 Gy s⁻¹ for the sample volume (10×10×10 mm³). The precursor solution was obtained by dissolving PEG DA in 0.6 mL deionized (D.I.) water; the volume ratio was 1/1, 1/3, 1/5, 1/10, 1/25 or 1/30. The solution contained no photoinitiator and the photopolymerization was achieved by exposure to the X-ray beam for 30, 60, 90, or 180 seconds.

### Characterization of the Synthesized Hydrogels

The characterization included FTIR with a Perkin-Elmer Spectrum One FTIR instrument (the substrate being a silicon wafer) and ¹H-nuclear magnetic resonance (NMR) spectroscopy with a Varian Unity Inova 500 MHz spectrometer. A 99.8% pure DMSO-d₆ solution was used as the reference. The following peaks consistent with the proposed structure of PEG DA-based hydrogel were observed: δ_{H} (500 MHz; DMSO-d₆, ppm): 2.4-2.6 (4H, —C(O)CH₂CH₂—), 3.7, (4H, —OCH₂CH₂—).

The surface morphology of our hydrogels was examined by scanning electron microscopy (SEM, JEOL, JSM-5600, Tokyo, Japan).

We analyzed the degradation of the synthesized hydrogels by first measuring their dry weight (Wi) after permanence for at least 24 hours after photopolymerization in a vacuum oven. The dried hydrogels were then incubated in 50 mL of PBS solution at 37° C.; the PBS solution was replaced daily. After a certain time, the hydrogels were removed, rinsed with PBS solution, vacuum-dried and weighed obtaining a dry weight value Wd. The relative weight loss (%) was calculated as (Wi−Wd) Wi⁻¹.

To determine the equilibrium swelling ratio of the hydrogels, their dry weight was measured immediately after photopolymerization, then they were allowed to swell in PBS at 37° C. for 1, 2, 3, 4, 5, 6 or 7 weeks; the PBS solution was again replaced daily. After the swelling period, the samples were rinsed with PBS, and the swollen hydrogel weight (Ws) was measured. The swelling ratio (Q) was calculated as Q=Ws Wd⁻¹.

### Molecule Inclusion in the Hydrogels

The molecules were heparin or PEI. In the first case, 300 mg of heparin sodium salt was dissolved in 3 mL D.I. water while stirring. The negatively charged heparin solution was added to the PEG DA solution (with a volume ratio of 1/25) reaching a concentration of 0.01, 0.02, 0.05, 0.1, 0.2 or 0.5 mg mL⁻¹ and X-ray irradiated for 90 seconds.

For the inclusion of PEI-based nanoparticles, 10 mg of branched PEI with an average Mw of 25,000 was added to 10 mL of D.I. water. The solution was then filtered with a 0.2 μm Millipore (Billerica, Mass.) instrument and stored at 4° C. A nucleic acid (plasmid DNA or siRNA: 1 μg) was then diluted in 100 μL D.I. water and vortexed. After about 1 minute, the PEI and nucleic acid solutions were mixed and vortexed for 30 minutes. The N/P ratios for PEI/DNA and PEI/siRNA nanoparticles (defined as PEI nitrogen/nucleic acid phosphate (N/P)) were 10/1 and 8/1, respectively. The PEI-based nanoparticle solution was then mixed with 1/25 volume ratio PEG DA solution and photopolymerized by 90 seconds of X-ray irradiation.

Electron micrographs were obtained with a high-resolution transmission electron microscope, HRTEM, JEOL JEM-2100F. The samples were prepared by depositing 10 μL of PEI/DNA or PEI/siRNA nanoparticle solution on a carbon-coated copper grid and air-drying.

### Cytotoxicity of the Hydrogels

Human HT-1080 fibroblasts (ATCC, Manassas, Va.) were grown in Dulbecco's modified Eagle's medium (DMEM, Biosource, Rockville, Md.) with 10% fetal bovine serum, 100 U mL⁻¹ penicillin, and 100 μg mL⁻¹ streptomycin at 37° C. in a humidified 5% CO₂ atmosphere. 10⁵ HT-1080 cells were seeded in each one of the wells of a 24-well plate and fed with complete DMEM for 12 hours. The cells were then exposed to PEG DA and PEG DA-based hydrogels at different concentrations (15.5, 18.6, 23.3, 46.6, 93.3 and 155.3 mg mL⁻¹). The exposures were performed with different PEG DA/D.I. water volume ratios (1/3, 1/5, 1/10, 1/20, 1/25 and 1/30), and different heparin concentrations (0.01, 0.02, 0.05, 0.100, and 0.500 mg mL⁻¹) and performed for 24 hours. The CellTiter 96® AQueous one solution cell proliferation assay system with the tetrazolium compound (3-(4,5-dimethylthiazol-2-yl)-5-(3-carboxymethoxyphenyl)-2-(4-sulfophenyl)-2H-tetrazolium, inner salt; MTS) was used to measure the mammalian cell survival and cell proliferation. The optical density (OD) value of formazan at 490 nm quantified the cell viability. The normalized cell viability was calculated by comparing the absorbance of untreated cells to that of cells exposed to PEG DA or to hydrogels.

Effects on Cells of Hydrogel-Mediated Nanoparticles for Plasmid DNA or siRNA Transfection

Cells of the same type as those above were seeded and incubated in DMEM with 100 U mL⁻¹ penicillin, 10% FBS, and 100 mg mL⁻¹ streptomycin for 12 hr before transfection. Subsequently, a porous polyester Transwell™ insert (Corning, N.Y.) with pore size of 1.0 μm was placed above the cell monolayer (in one well of a 6-well dish) to separate the hydrogels with PEI-based nanoparticles (Transwell™ insert) and the target HT-1080 cells (6-well dish). The 3.5 mL of complete DMEM medium was added to the culture without removing the Transwell™ insert; the complete DMEM medium solution was replaced daily. For the siRNA tests, we used recombinant HT1080 cells with the constitutive EGFP and luciferase expression as described in the literature. The transfected cells were then directly observed by a confocal microscope (Olympus IX 70, Olympus). The cells transfected with PEI-based nanoparticles were stained overnight by propidium iodide (PI, Molecular Probes, Eugene, Oreg.) to label the nuclei.

### Analysis of Reporter Expression

The PEI/plasmid DNA or PEI/siRNA nanoparticles were incorporated in the PEG DA-based hydrogel, and their release was evaluated by measuring EGFP intensities over one week. Flow cytometry analysis of EGFP-transfected cells was conducted with a benchtop system (FACSCalibur, Becton Dickinson) equipped with a 488 nm argon laser and a band-pass filter at 505-530 nm to detect EGFP. Untransfected cells were used as the control. The cells were appropriately gated by forward and side scatters, and 10,000 events per sample were collected. The assays of gene expression and gene silencing were quantitatively analyzed over one week.

### Animal Tests

All procedures involving animals were approved by Academia Sinica Institutional Animal Care and Utilization Committee (AS IACUC). BALB/cByJNarl mice (20-25 g) were provided by National Laboratory Animal Center (Taiwan). All mice were housed in individually ventilated cages (five per cage) with wood chip bedding and kept at 24±2° C. with a humidity of 40%-70% and a 12-hour light/dark cycle.

### In Vivo Hydrogels Photopolymerization and Biocompatibility

We injected 50 μL of PEG DA solution (with 1/25 volume ratio with respect to PBS) into the subcutaneous mouse tissue. The mice were placed on a movable stage for X-ray irradiation lasting 800 milliseconds. During X-ray irradiation the mice were kept under anesthesia using 1% isoflurene in oxygen. In vivo biocompatibility was examined on day seven to evaluate the immune responses of the epithelial cells overlying the either PEG DA or in situ photopolymerization of PEG DA-based hydrogel in subcutaneous mouse tissue. After sectioning, tissue slice sections of 10-15 μm thickness from each animal were diagnosed on the basis of haematoxylin and eosin (H&E, Sigma-Aldrich, Mo., USA) staining, and imaged with a Nikon ECLIPSE TS100 microscope.

### Tissue Slice Preparation

After synthesizing our hydrogel in vivo for 7 days, the mice (weight approximately 20-25 g) were sacrificed by intramuscular injection of Zoletil 50 (50 mg kg⁻¹; Virbac Laboratories, Carros, France). Subcutaneous tissue portions removed were immersed in the 3.7% paraformaldehyde for 24 hours. After fixation, the tissue portions were washed by PBS solution three times for 1 hour. All tissues were dehydrated by subsequent immersions in ethanol solutions, from low to high concentration, and then embedded in paraffin. Tissue specimens were sliced to 10 μm thickness and immersed in Xylene three times for 5 minutes to remove the remaining wax. Afterwards, the specimens were H&E stained for optical microscopy imaging.

### Gold Nanoparticles Inclusion in Hydrogel

The bare Au nanoparticle solution of 20 mM was added to the PEG DA solution (with volume ratio of 1/25) reaching a concentration of 0.67 mM and X-ray irradiated for 30 seconds. The Au nanoparticle solution was prepared and characterized following a previously developed method of one-pot synthesis by intense X-ray irradiation.

Although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for preparing a hydrogel, comprising the steps of:
injecting a precursor with at least two alkene groups into a predetermined portion;
injecting at least one specie into the predetermined portion, wherein the specie is selected from the group consisting of nucleic acid and adhesion agent; and
performing an X-ray irradiation on the predetermined portion to induce a polymerization reaction of the precursor to form a porous hydrogel with the specie embedded inside the porous hydrogel.

2. The method for preparing a hydrogel of claim 1, wherein the X-ray irradiation induces a 3-D polymerization reaction.

3. The method for preparing a hydrogel of claim 1, wherein the polymerization reaction is performed without using a chemical polymerization initiator.

4. The method for preparing a hydrogel of claim 1, wherein the polymerization reaction is performed for tens of seconds.

5. The method for preparing a hydrogel of claim 1, further comprising a step of adding gold particles into the predetermined portion.

6. The method for preparing a hydrogel of claim 1, wherein the precursor is poly(ethylene glycol) diacrylate.

7. The method for preparing a hydrogel of claim 1, wherein the nucleic acid is DNA or RNA.

8. The method for preparing a hydrogel of claim 1, wherein the nucleic acid is injected into the predetermined portion with a nucleic acid carrier.

9. The method for preparing a hydrogel of claim 8, wherein the nucleic acid carrier is polyethylenimine.

10. The method for preparing a hydrogel of claim 1, wherein the adhesion agent is an extracellular matrix.

11. The method for preparing a hydrogel of claim 1, wherein the extracellular matrix is selected from the group consisting of heparin, alginate, collagen, hyaluronan and the combination thereof.

12. The method for preparing a hydrogel of claim 1, wherein the predetermined portion is inside a live system.

13. The method for preparing a hydrogel of claim 12, further comprising a step of removing an reacted precursor by metabolism of the live system.

14. The method for preparing a hydrogel of claim 1, wherein the predetermined portion is outside a live system.

15. The method for preparing a hydrogel of claim 14, further comprising a step of injecting the porous hydrogel with the specie into a predetermined portion of a live system.

16. The method for preparing a hydrogel of claim 1, wherein the precursor includes at least two diacrylate groups.

17. The method for preparing a hydrogel of claim 1, wherein the precursor has a number average molecular weight of 258, 575, 700, 2000 or 60000.

18. A method for preparing a hydrogel, comprising the steps of:
injecting a precursor with at least two alkene groups into a predetermined portion inside a live system; and
performing an X-ray irradiation on the predetermined portion to induce a polymerization reaction of the precursor to form a porous hydrogel.

19. The method for preparing a hydrogel of claim 18, wherein the X-ray irradiation induces a 3-D polymerization reaction.

20. The method for preparing a hydrogel of claim 18, wherein the polymerization reaction is performed without using a chemical polymerization initiator.

21. The method for preparing a hydrogel of claim 18, wherein the polymerization reaction is performed for tens of seconds.

22. The method for preparing a hydrogel of claim 18, further comprising a step of adding gold particles into the predetermined portion.

23. The method for preparing a hydrogel of claim 18, wherein the precursor is poly(ethylene glycol) diacrylate.

24. The method for preparing a hydrogel of claim 18, further comprising a step of injecting at least one specie into the predetermined portion, wherein the specie is embedded inside the porous hydrogel after the polymerization reaction.

25. The method for preparing a hydrogel of claim 24, wherein the specie is selected from the group consisting of nucleic acid and adhesion agent.

26. The method for preparing a hydrogel of claim 25, wherein the nucleic acid is DNA or RNA.

27. The method for preparing a hydrogel of claim 25, wherein the nucleic acid is injected into the predetermined portion with a nucleic acid carrier.

28. The method for preparing a hydrogel of claim 27, wherein the nucleic acid carrier is polyethylenimine.

29. The method for preparing a hydrogel of claim 25, wherein the adhesion agent is extracellular matrix.

30. The method for preparing a hydrogel of claim 29, wherein the extracellular matrix is selected from the group consisting of heparin, alginate, collagen, hyaluronan and the combination thereof.

31. The method for preparing a hydrogel of claim 18, wherein the precursor includes at least two diacrylate groups.

32. The method for preparing a hydrogel of claim 18, wherein the precursor has a number average molecular weight of 258, 575, 700, 2000 or 60000.
